(19) United States
(12) Patent Application Publication
(10) Pub. No.: US 2006/0115424 A1
(43) Pub. Date: Jun. 1, 2006

(54) Combination therapy of oxaliplatin and radioactively doped particles treating cancer

(76) Inventor: Bruce Nathaniel Gray, Claremont (AU)

Correspondence Address: MERCHANT & GOULD PC, P.O. BOX 2903, MINNEAPOLIS, MN, 55402-0903, US

(21) Appl. No.: 10/525,298

(22) PCT Filed: Jul. 2, 2004
(86) PCT No.: PCT/AU04/00893

§ 371 Date: Aug. 15, 2005

(30) Foreign Application Priority Data
Jul. 2, 2003 (AU) 20003903387

(51) Int. Cl. A61K 51/00; A61K 31/7072; A61K 31/525; A61K 31/513; A61K 31/4745; A61K 31/28; A61K 39/395

(52) U.S. Cl. 424/1.11; 514/251; 514/283; 514/49; 514/269; 514/492; 424/155.1

(57) ABSTRACT

A method of treating cancer in a patient comprising administering to the patient an amount of oxaliplatin in combination with radioactively doped particle, characterised in that the two therapies when introduced into the patient have a synergistic anticancer effect.

### FIELD OF THE INVENTION

The present invention provides an improved method for treating cancer developed from the identification of an unexpected synergistic combination of known cancer therapies. It also relates to a therapeutic combination, which produces an unexpectedly greater treatment efficacy than each cancer therapy when used in the absence of the other therapy. The invention also relates to the use of the therapeutic combination described herein in the preparation of a medicament for the treatment of cancer.

### BACKGROUND ART

Cancer is now the second leading cause of death in the United States and is a disease characterized by an abnormal proliferation of cell growth known as a neoplasm. Malignant cancers, in particular, can result in a serious disease state, which may threaten life. Significant research efforts and resources have been directed toward the elucidation of anticancer measures, including chemotherapeutic and radiotherapeutic agents, which are effective in treating patients suffering from cancer. Effective anticancer agents include those that inhibit or control the rapid proliferation of cells associated with neoplasms, those that effect regression or remission of neoplasms, and those that generally prolong the survival of patients suffering from neoplasia. The terms neoplasia, malignant neoplasia, neoplastic growth and cancer are used interchangeably throughout this document.

Of the vast forms of malignant neoplasms colorectal cancer is one of the most common. The liver is a dominant site of metastatic spread of colorectal cancer as a result of the portal venous drainage of the gut and is the main cause of death in these patients. Treatment of such disease states is usually achieved with one or a combination of four therapies: surgery, chemotherapy, radiotherapy and immunotherapy.

Surgery involves the bulk removal of diseased tissue. When tumour growth is recognized, excision of the tumour mass by surgery is regarded as the therapy of choice. In a minority of patients with liver metastases some form of local ablation, such as cryotherapy or radiofrequency ablation, can also offer the potential for long-term cure. However, these approaches, while producing satisfactory results as a general measure, are effective only for patients with tumours at an early stage of development. They cannot be used in the liver, for example, where the vast majority of the liver is covered with multiple primary or secondary cancers.

Chemotherapy may involve the use of one or more anticancer drugs either with or without other cancer agents such as biologic modifying agents of which antibodies targeting the epidermal growth factor (EGF) or vascular endothelial growth factor (VEGF) are examples. For the purposes of this document “chemotherapy” means any combination of these agents. The major classes of anticancer drugs include alkylating agents, antimetabolites and antagonists, and a variety of miscellaneous agents (see Haskell, C. M., ed., (1995) and Dorr, R. T. and Von Hoff, D. D., eds. (1994)).

Of the alkylating agents, one such agent is oxaliplatin (OXA). Although the exact mechanism of action of OXA is yet not fully understood, it is believed to inhibit DNA synthesis. The primary use of OXA is in colorectal cancer. However, it may also be used to treat other cancers such as breast, gastric, lung, pancreatic and prostate cancers. Side effects associated with the use of OXA include numbness or tingling in hands and feet due to its effect on the nerve endings; temporary reduction in bone marrow function, resulting in anaemia, risk of bruising or bleeding, nausea and diarrhoea. Less common side effects include laryngeal spasm, allergic reactions, such as skin rashes and itching, and mouth ulcers.

Among the many antimetabolites that have been developed and clinically tested are methotrexate, 5-fluorouracil (5-FU), floxuridine, cytarabine, 6-mercaptopurine, 6-thioguanine, deoxycoformycin, fludarabine, 2-chlorodeoxyadenosine, and hydroxyurea. The compound 5-FU is possibly the most widely used anticancer drug in the world. 5-FU has been used clinically in the treatment of malignant tumours and cancer, including, for example, carcinomas, sarcomas, skin cancer, cancer of the digestive organs and liver, and breast cancer. 5-FU, however, causes serious adverse reactions such as nausea, alopecia, diarrhoea, stomatitis, leukocytic thrombocytopenia, anorexia, pigmentation, and edema. Further, as 5-FU is highly toxic, it is sometimes impossible to administer the compound over a prolonged period of time and therefore to achieve the desired curing effect.

Leucovorin (LV) addition to 5-FU approximately doubles the response rates in patients with gastrointestinal neoplasms. Currently, LV addition to 5-FU therapy is currently very commonly used in the United States.

While the combination of chemotherapy agents such as OXA, 5-FU and LV (the combination commonly known, as FOLFOX) has improved chemotherapy regimens there are still significant problems with the use of such agents. Among the problems currently associated with the use of such agents to treat cancer are the high doses of agent required, toxicity to normal cells, i.e. lack of selectivity, immunosuppression, second malignancies and drug resistance. Another side effect associated with such therapies is the toxic effect of the chemotherapeutic agents on the normal host tissues that are the most rapidly dividing such as the bone marrow, gut mucosa and the cells of the lymphoid system. The agents also exert a variety of other adverse effects, including neurotoxicity, negative effects on sexuality and gonadal function, and cardiac, pulmonary, pancreatic and hepatic toxicities; vascular and hypersensitivity reactions, and dermatological reactions.

The clinical usefulness of a chemotherapeutic agent may also be severely limited by the emergence of malignant cells resistant to that drug. In some cases, resistance to one drug may confer resistance to other biochemically distinct drugs. In this respect amplification of the gene encoding thymidylate synthase is related to resistance to treatment with 5-fluoropyrimidines.

In summary, chemotherapy has not made a dramatic impact on the treatment of cancer.

Radiotherapy has been used as an alternative to chemotherapy and usually relies on treatment through external beam technologies or through locally administering radioactive materials to patients with cancer in a technique known as brachytherapy. Examples of brachytherapy are where the radioactive materials have been incorporated into small particles, seeds, wires and similar related configurations that can be directly implanted into the cancer. When radioactive particles are administered into the blood supply of the target organ the technique has become known as Selective Internal Radiation Therapy (SIRT). Generally, the main form of application of SIRT has been its use to treat cancers in the liver. Liver cancer is particularly suited to treatment with SIRT due to the dual blood supply of the liver, which allows targeting of the radioactive particles to cancers within the liver when the radioactive particles are administered into the hepatic artery.

There are many potential advantages of SIRT over conventional, external beam radiotherapy. Firstly, the radiation is delivered preferentially to the cancer within the target organ. Secondly, the radiation is slowly and continually delivered as the radionuclide decays. Thirdly, by manipulating the arterial blood supply with vasoactive substances, it is possible to enhance the percentage of radioactive particles that go to the cancerous part of the organ, as opposed to the healthy normal tissues. This has the effect of preferentially increasing the radiation dose to the cancer while maintaining the radiation dose to the normal tissues at a lower level (Burton, M. A. et al. (1988) Europ. J. Cancer Clin. Oncol. 24(8), 1373-1376).

When microparticles or other small particles are administered into the arterial blood supply of a target organ, it is desirable to have them of a size, shape and density that results in the optimal distribution within the target organ.

For radioactive particulate material to be used successfully for the treatment of neoplastic growth, the radiation emitted should be of high energy and short range. This ensures that the energy emitted will be deposited into the tissues immediately around the particulate material and not into tissues that are not the target of the radiation treatment. In this treatment mode, it is desirable to have high energy but short penetration beta-radiation, which will confine the radiation effects to the immediate vicinity of the particulate material. There are many radionuclides that can be incorporated into microparticles that can be used for SIRT. Of particular suitability for use in this form of treatment is the unstable isotope of yttrium (Y-90). Yttrium-90 decays with a half-life of 64 hours by emitting high energy pure beta radiation. However, other radionuclides may also be used in place of Y-90 of which isotopes of holmium, samarium, iodine, iridium, phosphorus, rhenium are some examples.

The technique of SIRT has been previously reported (see, for example, Chamberlain M, et al (1983) Brit. J. Surg., 70: 596-598; Burton M A, et al (1989) Europ. J. Cancer Clin. Oncol., 25, 1487-1491; Fox R A, et al (1991) Int. J. Rad. Oncol. Biol. Phys. 21, 463-467; Ho S et al (1996) Europ J Nuclear Med. 23, 947-952; Yorke E, et al (1999) Clinical Cancer Res, 5 (Suppl), 3024-3030; Gray B N, et al. (1990) Int. J. Rad. Oncol. Biol. Phys, 18, 619-623). Treatment with SIRT has been shown to result in high response rates for patients with neoplastic growth in particular with colorectal liver metastases (Gray B. N. et al (1989) Sure. Oncol, 42, 192-196; Gray B, et al (1992) Aust NZ J Surgery, 62, 105-110; Gray B N et al. (2000) GI Cancer, 3(4), 249-257; Stubbs R, et al (1998) Hepato-gastroenterology Suppl II, LXXVII). Other studies have shown that SIRT therapy can also be effective in causing regression and prolonged survival for patients with primary hepatocellular cancer (Lau W, et al (1994) Brit J Cancer 70, 994-999; Lau W, et al. (1998) Int J Rad Oncol Biol Phys. 40, 583-592). Although SIRT is effective in controlling the liver disease, it has no effect on extra-hepatic disease.

Recently, clinicians have tried to improve the effectiveness of cancer treatment by combining two or more anticancer therapies into a single therapeutic regimen. One example of such combination therapy is demonstrated by the randomised clinical trial carried out by Gray et al where they compared treatment of cancer by floxuridine either with or without the addition of a single dose of radioactive microparticles (Gray et al (2001) Annals of Oncology 12: 1711-1720). This study has shown that the addition of radioactive microparticles increased the response rate from 17.6% to 44% and the time to disease progression from 9.7 months to 15.9 months. An important finding from this trial was that although most patients eventually succumbed to their disease, the liver metastases were not the primary cause of death for most patients treated with SIRT.

Combination therapies now being tested use agents with dissimilar mechanisms of action, based on the rationale that targeting two independent pathways will result in enhanced cytotoxicity, whether additive or synergistic. The results of these experiments are entirely unpredictable as the use of two entirely different therapies usually means that each therapy works independently of the other and thus would not be expected to interact to improve the other.

It would be advantageous to show that combining chemotherapy with other forms of cancer therapy, such as brachytherapy using SIRT, resulted in an improved outcome for cancer patients. It is well recognised that the outcome measure of ‘response’ is a measure of the ability of the treatment to cause regression of a cancer and that prolongation of the time a cancer is held in remission, known as ‘time to disease progression’, is a measure of particular benefit. There is described herein a process which provides such advantages.

### SUMMARY OF THE INVENTION

The present invention concerns an unexpected combination of known anticancer therapies, which provides unexpected synergistic anticancer effect. Accordingly, the present invention provides a method that has utility in the treatment of various forms of cancer and tumours, particularly in the treatment of primary and secondary liver cancer and, more specifically, secondary liver cancer deriving from the gastrointestinal tract such as secondary liver cancer deriving from colorectal cancer.

It is to be understood that the SIRT described herein should not be limited to radioactive microparticles, but may be extended to any radioactive particles or materials of any sort, of which targeted antibodies labelled with a therapeutic radioactive material is one example, that are suitable for use in the treatment methods described herein.

Accordingly, the present invention provides a method of treating cancer in patients by administering to the subject an amount of OXA in combination with SIRT, wherein a synergistic anticancer effect results. Although OXA may be the only chemotherapeutic agent employed in the method, it will be appreciated that other chemotherapeutic agents may be used in the method. Preferably 5-FU and LV are included in combination with OXA. Other chemotherapeutic agents that may be employed in the method in addition to 5-FU and LV include systemic chemotherapy drugs such as irinotecan or capecitabine. Further, the method may also include a step of treating the patient with anti-angiogenesis factors, i.e. agents that inhibit the blood supply to cancers. Still further, other anticancer agents such as antibodies targeted against a variety of cancer cells or the blood vessels supplying the cancer cells, for example antibodies targeting EGF and VEGF, may also be used. Preferably, the method is used for treating a patient with colorectal liver metastases.

The invention further provides a synergistic combination of anticancer agents comprising an effectively therapeutic amount of OXA chemotherapy and an amount of radionuclide-doped agents suitable for SIRT to effectively treat cancer. Preferentially, oxaliplatin chemotherapy is combined with 5-fluorouracil and leucovorin or other agents, of which all possible combinations are known collectively as ‘oxaliplatin based therapy’, or OBT, to enhance the chemotherapeutic effect. For example, the FOLFOX combination may be used with the addition of other anticancer agents such as other chemotherapeutic drugs and agents that use biologic or immunologic targeting.

There are many other anticancer agents that may be used in combination with oxaliplatin and which are hereby included within the definition of ‘oxaliplatin based therapy’. Examples of these include fluorouracil-based drugs, irinotecan, monoclonal antibody targeted therapy and anticancer agents directed against vascular endothelial growth factor (VEGF) or epidermal growth factor (EGF).

The invention also provides for the use of effective amounts of OXA or oxaliplatin based therapy and an amount of radionuclide-doped particles suitable for SIRT to effectively treat cancer in the preparation of a medicament for the treatment of cancer generally and in particular primary liver cancer, secondary liver cancer, secondary liver cancer deriving from the gastrointestinal tract, and more specifically secondary liver cancer deriving from colorectal cancer. Also, cancer of the brain, cancer of the kidney, cancer in other soft tissues, and bone sarcomas.

The present invention further provides a synergistic anticancer combination of anticancer agents, comprising an effective anticancer amount of OXA or oxaliplatin based therapy and an amount of radionuclide-doped particles suitable for use in SIRT for treatment of a neoplastic growth. This combination may be used to treat all forms of primary or secondary liver cancer, preferably secondary gastrointestinal cancer and, more preferably, the combination is used to treat patients with colorectal liver metastases.

The invention also relates to pharmaceutical compositions comprising an effective anticancer amount of OXA or OBT and an amount of radionuclide-doped particles suitable for use in SIRT for the treatment of cancer. Preferably, the pharmaceutical composition is prepared for use in treating a patient with colorectal liver metastases. In addition to the pharmaceutical composition including OXA or OBT it may include one or more alternate chemotherapeutic agents and/or anti-angiogenesis agents and/or other anti cancer agents. Such agents will include but will not be limited to 5-FU, LV, irinotecan, capecitabine and antibodies directed against EGF and VEGF.

The invention still further relates to the use of an effective anticancer amount of OXA or OBT and an amount of radionuclide-doped particles suitable for use in SIRT, for manufacture of a medicament for treating cancer in a cancer patient. Preferably, the medicament is prepared for use in treating a patient with colorectal liver metastases. In addition the medicament manufactured according to this aspect of the invention may also include one or more alternate chemotherapeutic agents and/or anti-angiogenesis factors. Such agents will include but will not be limited to 5-FU, LV, irinotecan, capecitabine and antibodies directed against EGF and VEGF.

Other aspects and advantages of the invention will become apparent to those skilled in the art from a review of the ensuing description.

### DETAILED DISCLOSURE OF THE INVENTION

General

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in the specification, individually or collectively, and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

All references cited, including patents or patent applications are hereby incorporated by reference. No admission is made that any of the references constitute prior art.

Throughout this specification, unless the context requires otherwise, the word “comprise”, or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout.

### Description of Preferred Embodiments

Surprisingly, applicants have found that the co-administration of systemic chemotherapy and SIRT to a patient with liver cancer, potentiates the effect of the radiation from SIRT on the liver cancer, and also has a beneficial effect on extra-hepatic disease.

Accordingly, the present invention provides a method of treating a cancer patient by administering to the patient an amount of OXA or OBT effective to treat the cancer, in combination with SIRT, wherein a synergistic anticancer effect results.

Although OXA may be the only chemotherapeutic agent employed in the method, it will be appreciated that other chemotherapeutic agents may be used in the method. Preferably both 5-FU and LV, are included in combination with OXA. For ease of description the following disclosure is framed in terms of using OXA in combination with 5-FU and LV as this is a common combination used for the treatment of malignant neoplasias. The present invention should not be read as being limited to only the use of such a combination in the method, but includes only the use of OXA in the method or the use of OXA and 5-FU or the use of OXA with other chemotherapeutic, biologic or immunologic agents. All such combinations are referred to here as oxaliplatin based therapy or OBT.

Other chemotherapeutic agents that may be employed in the method either in addition to 5-FU and LV include systemic chemotherapy drugs such as irinotecan or capecitabine. Further, the method may also include a step of treating the patient with anti-angiogenesis factors, i.e. drugs that inhibit blood supply of cancers. Further, other anticancer agents such as antibodies targeted against a variety of cancer cells, or the blood vessels supplying the cancer cells, may also be used. Antibodies targeting EGF and VEGF are examples of such antibodies. Preferably, the method is used for treating a patient with colorectal liver metastases.

The present invention provides a method of treating cancer. Cancers for which the present invention will be particularly useful include, without limitation, primary liver cancer and secondary liver cancer deriving from the gastrointestinal tract, and more specifically secondary liver cancer deriving from colorectal cancer. Also, cancer of the brain, cancer of the kidney, cancer in other soft tissues, and bone sarcomas.

In the method of the present invention, 5-FU, LV and OXA or OBT is administered to a patient in combination with SIRT, such that a synergistic anticancer effect is produced. A “synergistic anticancer effect” refers to a greater-than-additive anticancer effect that is produced by a combination of chemotherapeutic drugs and SIRT, which exceeds that which would otherwise result from individual therapy associated with either therapy alone. Treatment with 5-FU, LV and OXA in combination with SIRT unexpectedly results in a synergistic anticancer effect by providing a greater effect than would result from use of either of the anticancer agents alone.

In the method of the present invention, administration of 5-FU, LV and OXA “in combination with” SIRT refers to co-administration of the three anticancer treatments. Co-administration may occur concurrently, sequentially, or alternately. Concurrent co-administration refers to administration of 5-FU, LV and OXA and SIRT at or about the same time. For concurrent co-administration, the courses of treatment with 5-FU, LV and OXA and with SIRT may also be run simultaneously., For example, a single, combined formulation of 5-FU, LV and OXA, in physical association with SIRT, may be administered to the subject.

Generally SIRT is administered on only one or two occasions whereas treatment with 5FU, LV and OXA are administered at or about the time of SIRT and are continued as an ongoing treatment.

In the method of the present invention, 5-FU, LV and OXA therapy and SIRT also may be administered in separate, individual treatments that are spaced out over a period of time, so as to obtain the maximum efficacy of the combination. When spaced out over a period of time, administration of 5-FU, LV and OXA is preferably given to a patient for a period of time such as 1 to 10 days, but more preferably about 3 to 5 days. This cycle may be repeated as many times as necessary and as long as the subject is capable of receiving said treatment.

As used herein “treatment” includes:
(i) preventing a disease, disorder or condition from occurring in a patient who may be predisposed to the disease, disorder and/or condition but has not yet been diagnosed as having it;
(ii) inhibiting the disease, disorder or condition, i.e., arresting its development; or
(iii) relieving the disease, disorder or condition, i.e., causing regression of the disease, disorder and/or condition.

In the method of the present invention, cancer is treated in a patient in need of treatment by administering to the patient an amount of a combination of 5-FU, LV and OXA effective to treat a cancer in combination with a sufficient amount of SIRT to treat a cancer, wherein a synergistic anticancer effect results.

The patient is preferably a mammal and is most preferably a human.

5-FU, LV and OXA Chemotherapy

In the method of the present invention, an amount of 5-FU, LV and OXA that is “effective to treat the cancer” is an amount that is effective to ameliorate or minimize the clinical impairment, growth or symptoms of the cancer, in either a single or multiple dose of 5-FU, LV and OXA when combined with SIRT. For example, the clinical impairment or symptoms of the cancer may be ameliorated or minimized by diminishing any pain or discomfort suffered by the patient; by extending the survival of the patient beyond that which would otherwise be expected in the absence of such treatment; by inhibiting or preventing the development or spread of the cancer, or by limiting, suspending, terminating, or otherwise controlling the maturation and proliferation of cells in the cancer. Notably, the amounts of 5-FU, LV and OXA effective to treat cancer in a patient in need of treatment will vary depending on the type of SIRT used, as well as the particular factors of each case, including the type of cancer, the stage of the cancer, the patient's weight, the severity of the patient's condition, and the method of administration. These amounts can be readily determined by the skilled artisan.

5-FU, LV and OXA treatment according to the present invention may be administered to a patient by known procedures, including, but not limited to, oral administration, parenteral administration (e.g., intramuscular, intraperitoneal, intravascular, intravenous, or subcutaneous administration), and transdermal administration. Preferably, the 5-FU, LV and OXA agents are administered parenterally.

SIRT Therapy

According to the invention the person skilled in the art will appreciate that SIRT may be applied by any of a range of different methods, some of which are described in U.S. Pat. Nos. 4,789,501, 5,011,677, 5,302,369, 6,296,831, 6,379,648, or WO applications 200045826, 200234298 or 200234300 (incorporated herein by reference). Accordingly, administration of radionuclide doped microparticles may be by any suitable means, but preferably by delivery via the relevant artery. For example, in treating liver cancer, administration is preferably by insertion of a catheter into the hepatic artery. Pre or co-administration of another agent may prepare the tumour for receipt of the particulate material, for example a vasoactive substance, such as angiotension-2 to redirect arterial blood flow into the tumour. Delivery of the particulate matter may be by single or multiple doses, until the desired level of radiation is reached.

The radionuclide doped microparticles need not be limited to any particular form or type of microparticle. So, for example, the radionuclide doped microparticles suitable for use in the invention may comprise any material capable of receiving a radionuclide such as through impregnation, absorbing, coating or more generally bonding the radionuclide with the microparticle or material used to carry the radionuclide.

In one particular form of the invention the microparticles are prepared as polymeric particles. In another form of the invention the microparticles are prepared as ceramic particles (including glass). In another, they are prepared from chitosan. In another they are formed of yttria. In another they are formed substantially from silicon. In another they are formed from proteins. In another they are formed from antibodies.

Where the microparticles are prepared as a polymeric matrix they will preferably have a stably incorporated radionuclide. More preferably the radionuclide will be incorporated by precipitation of the radionuclide as a salt. A description of such particles including methods for their production and formulation as well as their use is provided in co-owned European application number 200234300, of which the teachings therein are expressly incorporated herein by reference.

Where the particles are based on silicon the radionuclide will preferably be stably incorporated into the silicon matrix or within the pores or micropores of the matrix or coated onto the matrix.

Where the particles are based on yttria, the radionuclide will preferably be stably incorporated into the yttria matrix or coated onto the surface.

Where the microparticles are ceramic particles (including glass) the selected particles will usually possess the following properties:
(1) the particles will generally be biocompatible, such as calcium phosphate-based biomedical ceramics or glass, or aluminium-boro silicate glass, or silicate based glass.
(2) the particles will generally comprise a radionuclide that preferably emits radiation of sufficiently high energy and with an appropriate penetration distance in tissue, which are capable of releasing their energy complement within the tumour tissue to effectively kill the cancer cells and to minimize damage to adjacent normal cells or to attending medical personnel. The level of radiation activity of the ceramic or glass will be selected and fixed based upon the need for therapy given the particular cancer involved and its level of advancement. The ideal half-life of the radionuclides is somewhere between days and months. On the one hand, it is impractical to treat tumours with radionuclides having too short a half-life, this characteristic limiting therapy efficiency. On the other hand, in radiotherapy it is generally difficult to trace and control radionuclides having a long half-life.
(3) Third, the particles must be of a suitable size. The size of the particles for treatment depends upon such variables as the selected method of introduction into the tumour.

There are many processes for producing small granular ceramic or glass particles. One of these involves the introduction of small amounts of the ceramic particles passing through a high-temperature melting region. Ceramic spherules are yielded by surface tension during melting. After the solidification, condensation, collection and sorting processes, ceramic spherules of various sizes can be obtained. The particle size of ceramic spheroids can be controlled by the mass of granules introduced into the high-temperature melting region or can be controlled by collecting spheroids of various sizes through the selection of sedimentation time during liquid-sedimentation.

The ceramic or glass materials for preparing those particles can be obtained commercially or from ultra-pure ceramic raw materials if the commercial products do not meet specifications for one reason or another. The ceramic or glass particles for radiation exposure in this invention can be yielded by traditional ceramic processes, which are well known by those skilled in this art. The ceramic processes such as solid-state reaction, chemical co-precipitation, sol-gel, hydrothermal synthesis, glass melting, granulation, and spray pyrolysis can be applied in this invention for the production of specific particles.

The microparticles of the invention be they polymer, ceramic, glass or silicon based or other can be separated by filtration or other means known in the art to obtain a population of microparticles of a particular size range that is preferred for a particular use.

The radionuclide which is incorporated into the microparticles in accordance with the present invention is preferably yttrium-90, but may also be any other suitable radionuclide of which holmium, samarium, iodine, phosphorous, iridium and rhenium are some examples.

The amount of microparticles used in the method and which will be required to provide effective treatment of a neoplastic growth will depend on the radionuclide used in the preparation of the microparticles. By way of example, an amount of yttrium-90 activity that will result in an inferred radiation dose to the normal liver of approximately 80 Gy may be delivered. Because the radiation from SIRT is delivered as a series of discrete point sources, the dose of 80 Gy is an average dose with many normal liver parenchymal cells receiving much less than this dose. Alternate doses of radiation may be delivered depending on the disease state and the physician's treatment needs. Such variation of radiation doses obtained by altering the amount of microparticles used will be something that a skilled artisan will know how to determine.

The term microparticle is used in this specification as an example of a particulate material, it is not intended to limit the invention to microparticles of any particular shape or configuration. A person skilled in the art will, however, appreciate that the shape of the particulate material will preferably be substantially spherical, but need not be regular or symmetrical in shape and could be of any shape or size.

In a highly preferred form of the invention there is provided a method for treating cancer in a cancer patient, by administering an effective anticancer amount of 5-FU, LV and OXA or OBT as described above in combination with an amount of radionuclide-doped microparticles suitable for use in SIRT for treatment of cancer, wherein a synergistic anticancer effect results.

It has been shown that the beneficial effect of OBT can be enhanced by the addition of agents that target the blood vessels supplying tumours such as agents that inhibit angiogenesis. The present invention includes the addition of these agents when used together with OBT and SIRT.

In addition to the identified chemotherapeutic agents and radionuclide doped microparticles the invention may also include an effective treatment of immunomodulators and other agents as part of the therapy. Illustrative immunomodulators suitable for use in the invention are alpha interferon, beta interferon, gamma interferon, interleukin-2, interleukin-3, tumour necrosis factor, and the like.

The present invention further provides a synergistic combination of anticancer agents, comprising an effective anticancer amount of 5-FU, LV and OXA or OBT and an amount of radionuclide-doped microparticles suitable for use in SIRT for treatment of a neoplastic growth. The present invention provides a method that has utility in the treatment of various forms of cancer and tumours, but particularly in the treatment of primary liver cancer and secondary liver cancer and, more specifically, secondary liver cancer deriving from the gastrointestinal tract, and most specifically secondary liver cancer deriving from colorectal cancer. Preferably, the combination is prepared for use in treating a patient with colorectal liver metastases.

The invention also relates to pharmaceutical compositions comprising an effective anticancer amount of 5-FU, LV and OXA and an amount of radionuclide-doped microparticles suitable for use in SIRT for treatment of a neoplastic growth. Preferably, the pharmaceutical composition is prepared for use in treating a patient with colorectal liver metastases.

The invention still further relates to use of an effective anticancer amount of 5-FU, LV and OXA or OBT as described above and an amount of radionuclide-doped microparticles as described above suitable for use in SIRT, for manufacture of a medicament for killing neoplastic cells in a subject having neoplastic disease. The present invention provides a method that has utility in the treatment of various forms of cancer and tumours, but particularly in the treatment of primary liver cancer and secondary liver cancer and, more specifically, secondary liver cancer deriving from the gastrointestinal tract, and most specifically secondary liver cancer deriving from colorectal cancer. Preferably, the medicament is prepared for use in treating a patient with colorectal liver metastases.

### EXAMPLES

Further features of the present invention are more fully described in the following non-limiting example. It is to be understood, however, that this detailed description is included solely for the purposes of exemplifying the present invention. It should not be understood in any way as a restriction on the broad description of the invention as set out above.

Patients: Nine patients with colorectal liver metastases either with or without extra-hepatic metastases were enrolled in this study. Patients were between 45 and 70 years of age, had histologically proven colorectal adenocarcinoma, and unequivocal CT scan evidence of liver metastases that could not be treated by resection or any locally ablative technique.

Patients received systemic chemotherapy (5-FU, LV and OXA) with the addition of a single administration of SIR-Spheres® (Sirtex Medical Ltd). All patients had multiple liver metastases and were reviewed to confirm that the metastases were so advanced that they were unable to be treated by any form of local ablation.

Investigations: All patients underwent a pre-treatment spiral CT scan of the whole abdomen and either a CT scan of the chest or chest X-ray and blood tests to assess haematologic, renal and liver function and serum CEA.

Patients treated with SIRT underwent a trans-femoral hepatic angiogram to assess the arterial anatomy of the liver and to plan the subsequent administration of SIR-Spheres®. Patients treated with SIRT also underwent a nuclear medicine scan to estimate the amount of SIR-Spheres® that would pass through the liver and lodge in the lungs. This was performed by injecting technetium-99 labelled macro-aggregated albumin (MAA) into the hepatic artery at the time of the angiogram and measuring the radioactivity in the liver and lungs using a gamma camera. Areas of interest were drawn around the liver and lungs and the percentage of the MAA that lodged in the lungs was determined as a fraction of the total amount of MAA in both lungs and liver. This was recorded as a ‘percentage lung break-through’ in order to decide whether to reduce the amount of yttrium-90 activity to administer to the patient. Previous experiments had shown that a lung break-through percentage of >13% might result in radiation pneumonitis and should be accompanied by a reduction in the amount of yttrium-90 activity administered to the patient (Ho S et al (1996) Europ J Nuclear Med. 23, 947-952). This technique has been shown to be a reliable method for determining the subsequent distribution of SIR-Spheres®.

Patients were followed after trial entry with three monthly clinical evaluations and quality of life assessment (QoL), three-monthly CT scans of the abdomen were also carried out as were either a plain X-ray or CT scan of the chest. Further, regular monthly serologic tests of haematologic, liver and renal function and CEA were taken. Patients found to have obtained a complete (CR) or partial (PR) response on CT scan had a second confirmatory CT scan at not less than 4 weeks after the initial scan that showed the response.

Recording of Response and Toxicity: Response was determined using RECIST criteria (Therasse P et al (2000) J Natl Cancer Inst 92, 205-216). The RECIST criteria were developed with particular application for reporting the results of phase 2 trials and result in response outcomes that are very similar to those using the conventional WHO method.

Toxicity was recorded on all patients using standard UICC recommendations for grading of acute and subacute toxicity criteria.

Protocol Treatment: Patients were treated with a combination of Oxaliplatin, 5-Fluorouacil, Leucovorin (FOLFOX-4) and SIR-spheres. Oxaliplatin 30 mg/m² or 60 mg/m², dependent on treatment group, was administered on day 1 of each cycle. Leucovorin 200 mg/m² followed by 5-fluorouracil 400 mg/m² as IV bolus and 600 mg/m² 5-Fluorouracil as 22-h continuous infusion were administered on days one and two of each cycle. Chemotherapy cycles were repeated at two weekly intervals and continued in patients until evidence of unacceptable toxicity, patient request or disease progression. Patients received a maximum of 12 cycles of protocol chemotherapy.

Patients received a single dose of SIR-Spheres® that was administered on either day two or day three of the first cycle of chemotherapy. The SIR-Spheres® was administered into the hepatic artery via a trans-femoral catheter that was placed using local anaesthetic. In patients where there was more than one hepatic artery supplying blood to the liver, the catheter was repositioned during administration and the total dose of SIR-Spheres® was divided into separate aliquots depending on the estimated volume of tumour being supplied by each feeding artery. Patients treated with SIRT were generally kept in hospital overnight and discharged home the following day.

Patients were treated with a dose of SIR-Spheres® that was calculated from the patient's body surface area and the size of the tumour within the liver according to the following equation;

$$Dose of SIR \text{-} {Spheres}^{®} in GBq = ( {BSA}^{*} - 0.2 ) + ( \frac{\% tumour involvement}{100} )$$

$${}^{*} BSA = body surface area measured in square metres$$

Non-Protocol Treatment: Once protocol treatment ceased, further cancer specific treatment, including non-protocol chemotherapy, was allowed to best manage patient care. All non-protocol cancer specific treatment was recorded in all patients. Other supportive, but not cancer specific treatment was allowed for patient management.

Results

Patients: Three patients (numbered 201002, 201003 and 605001) were treated at the initial Oxaliplatin dose level, 30 mg/m² and received between 1.3-3.2 GBq of SIR-Spheres®. All patients completed chemotherapy as per the protocol. Of the initial 3 patients, all showed evidence of response with reduction in tumour size on CAT scans.

Since protocol treatment was well tolerated at the first dose level a further six patients were treated at the higher dose level. Five of the nine patients have had follow up scans performed, and all five have recorded responses by RECIST criteria. Three of the other 4 patients that are awaiting follow up scans have had their serum CEA levels measured. All three show a reduced CEA level following treatment, indicating a biologic response to therapy. This means that effectively 100% of the evaluable patients so far show a positive response to treatment by the combination of FOLFOX and SIRT. In addition, there has been only one grade 4 toxicity event in all the patients treated so far, indicating the very acceptable toxicity profile of the therapy.

Discussion

Follow up of patients has revealed that an unusually large percentage of patients are responding to treatment with the SIRT+FOLFOX combination. The toxicity profile has been low in comparison with other chemotherapy regimens with only one patient experiencing a grade 4 toxicity event. These results are extra-ordinarily positive and far greater than is recorded for similar patients treated with FOLFOX alone where positive response rates rarely exceed 40%. Similarly, positive response rates for patients treated by SIRT alone are generally much lower, with one recent study reporting a 29% response rate.

SIRT is a form of localised brachytherapy. Brachytherapy is not used in combination with systemic chemotherapy as the brachytherapy is expected to adequately deal with localised disease. Furthermore, prior to the work described here, there has been no evidence that systemic chemotherapy using oxaliplatin-based chemotherapy can enhance the local effect of any form of brachytherapy, including SIRT. Therefore the outcome from treating patients with a combination of a local therapy such as SIRT together with a systemic chemotherapy regimen is unknown.

Until now there has been no evidence that combining a local therapy such as SIRT with a systemic chemotherapy regimen would result in any advantage over using either treatment alone. The experiment described above has shown for the first time that combining a local brachytherapy treatment (SIRT) with a systemic chemotherapy regimen (FOLFOX) does greatly improve the response rate of patients who are otherwise considered to be very difficult to treat. The fact that 100% of patients responded to treatment with the combination of SIRT plus FOLFOX is extraordinary and shows an unexpected synergy between the two modes of treatment.

1. A method of treating cancer in a patient comprising administering to the patient an amount of oxaliplatin in combination with radioactively doped particle, characterised in that the two therapies when introduced into the patient have a synergistic anticancer effect.

2. A method according to claim 1 wherein in addition to oxaliplatin other chemotherapeutic agents are also delivered to the patient with the oxaliplatin.

3. A method according to claim 2 wherein an effective anticancer amount of 5-FU and or LV are administered with oxaliplatin or a oxaliplatin based therapy'

4. A method according to claim 2 wherein other chemotherapeutic agents that may be employed in the method include systemic chemotherapy drugs such as irinotecan or capecitabine.

5. A method according to claim 1 wherein the patient is also treated with an anti-angiogenesis factor.

6. A method according to claim 1 wherein the patient is also treated with other anticancer agents including antibodies targeted against a cancer cells or the blood vessels supplying the cancer cells or antibodies targeting EGF and VEGF.

7. A therapeutic combination of anticancer agents comprising an effectively therapeutic amount of oxaliplatin and an amount of radionuclide-doped agent suitable for SIRT to effectively treat a cancer, wherein the two therapies when administered to a patient lead to a synergistic anticancer effect that is therapeutically more efficacious that either treatment when administered alone.

8. A therapeutic composition according to 7 wherein oxaliplatin is combined with 5-fluorouracil and leucovorin.

9. A therapeutic composition according to 8 wherein oxaliplatin is combined with other systemic chemotherapy drugs such as irinotecan or capecitabine.

10. A therapeutic composition according to 8 wherein oxaliplatin is combined with an anti-angiogenesis factor.

11. A therapeutic composition according to 8 wherein oxaliplatin is combined with an anticancer agent including antibodies targeted against a cancer cells or the blood vessels supplying the cancer cells or antibodies targeting EGF and VEGF.

12. (canceled)

13. The method according to claim 1 wherein the cancer is a primary or secondary cancer.
